(19) United States
(12) Patent Application Publication
(10) Pub. No.: US 2009/0286223 A1
(43) Pub. Date: Nov. 19, 2009

(54) Methods And Kits For In Situ Measurement of Enzyme Activity and Amount Using Single Measurement System

(75) Inventors: Sue Goo Rhee, Seoul (KR); Jong Seo Lee, Seoul (KR); Sung Won Bae, Seoul (KR); Chun Seok Cho, Bucheon-shi (KR); Jeong Woo Shin, Seoul (KR); Young Ha Lee, Seoul (KR); Sook Yeon Lee, Seoul (KR); Yun Hwa Kim, Seoul (KR); So Mi Oh, Seoul (KR)

Correspondence Address: SUGHRUE MION, PLLC, 2100 PENNSYLVANIA AVENUE, N.W., SUITE 800, WASHINGTON, DC, 20037, US

(73) Assignee: Ewha University-Industry collaboration Foundation, Seoul (KR)

(21) Appl. No.: 12/302,418

(22) PCT Filed: May 30, 2006
(86) PCT No.: PCT/KR2006/002082

§ 371 Date: Nov. 25, 2008

(51) Int. Cl. C12Q 1/70; G01N 33/53

(52) U.S. Cl. 435/5; 435/7.1

(57) ABSTRACT

Disclosed are methods and kits for measuring in situ the activity and amount of an enzyme in a sample using a single measurement system. The method includes the steps of: (a) contacting a sample to a capturing agent having the capacity to bind to the enzyme to be analyzed and immobilized on a solid matrix; (b) measuring the activity of the enzyme captured by the capturing agent; (c) contacting a detection antibody specific to the enzyme captured by the capturing agent; and (d) detecting an antigen-antibody complex formed in the step (c). There is no need for separate measurement system to measure the activity and amount of an enzyme, as well as the above methods and kits permit to measure the precise activity and amount of an enzyme simultaneously because the measurement of enzyme activity and amount is carried out in a single and same measurement system as to the same sample in almost simultaneous manner.

### BACKGROUND OF THE INVENTION

1. Field of the Invention

The present invention relates to methods and kits for measuring in situ the activity and amount of an enzyme in a single measurement system.

2. Description of the Related Art

The measurement of enzyme activity and amount is an essential experimental procedure in the field of enzyme biotechnology. Much research and study on the method for measurement of enzyme activity and amount has been already done. For example, Biuret reaction method (Itzhaki, R. F. et al., Anal. Biochem. 9:401-410(1964)), Lowry reaction method (Lowry, O. H. et al., J. Biol. Chem. 193:265-275(1951)), UV absorption measurement method (Warburg, O. et al., Biochem. Z. 310:384-421(1941)) and Dye binding method (Bradford, M. M. et al., Anal. Biochem. 72:248-254(1976)) have been already developed for analyzing the amount of enzymes

Methods for measuring the activity of an enzyme is largely classified into two approaches: stopped methods and continuous methods. The continuous methods include uncoupling methods and coupling methods. The methods for measuring the activity of an enzyme are described by Robert K. Scopes, Protein Purification, 2nd ed. Springer-Verlag New York Inc.(1988); Crabtree, B. et al., In Techniques in the life sciences, Vol. B211, pp. 1-37, Elsevier/North-Holland, Amsterdam.(1079); Scopes, R. K. Anal. Biochem. 49:73-87(1972); and Garcia-Carmona, F. et al., Anal. Biochem. 113:286-291(1981).

However, according to currently developed methods for enzyme assay, the activity and amount of an enzyme are measured in separate assay systems. According to such conventional measuring technologies, they cannot guarantee the utilization of identical samples for measuring both enzyme activity and amount and generally require large amount of samples because the activity and amount of an enzyme are measured independently. In addition, the conventional processes are very likely to be tedious due to different manipulations for enzyme activity and amount analysis and to demand a longer time for measurement. Furthermore, the conventional methods have the difficulty in discriminating the amount of active enzyme from that of inactive one.

Throughout this application, various patents and publications are referenced and citations are provided in parentheses. The disclosure of these patents and publications in their entities are hereby incorporated by references into this application in order to more fully describe this invention and the state of the art to which this invention pertains.

### DETAILED DESCRIPTION OF THIS INVENTION

The present inventors have made intensive researches to solve the demerits of the prior arts, and as a result developed methods and kits for measuring in situ measurement of enzyme activity and amount using a single measurement system.

Accordingly, it is an object of this invention to provide methods for measuring in situ the activity and amount of an enzyme in a single measurement system.

It is another object of this invention to provide kits for measuring in situ the activity and amount of an enzyme in a single measurement system.

Other objects and advantages of the present invention will become apparent from the following detailed description together with the appended claims and drawings.

In one aspect of this invention, there is provided a method for measuring in situ the activity and amount of an enzyme in a single measurement system, which comprises the steps of: (a) contacting a sample to a capturing agent having the capacity to bind to the enzyme to be analyzed and immobilized on a solid matrix; (b) measuring the activity of the enzyme captured by the capturing agent; (c) contacting a detection antibody specific to the enzyme captured by the capturing agent; and (d) detecting an antigen-antibody complex formed in the step (c).

The present invention relates to methods for measuring in situ the activity and amount of an enzyme in a sample using a single measurement system.

The term “in situ” used herein with reference to the present measurement methods means that the activity and amount of an enzyme are simultaneously measured by using the same enzyme immobilized by the capturing agent of the present single measurement system. In other words, according to the present invention, the activity and amount of enzyme can be measured in in situ manner since the phase of the enzyme to be analyzed will not be changed while being measured. In addition, the method of the present invention can be expressed as a simultaneous quantitating method to measure the activity and amount of an enzyme.

The term “single measurement system” used herein means that if a matrix (for example, microplate) on the surface of which a capturing agent is immobilized is used as a reaction system, this matrix can be used as it is to measure the activity and amount of an enzyme.

Any suitable samples can be analyzed using the method of the present invention. For example, the sample includes virus, bacterium, cell or tissue-derived extract, lysate or purified materials, blood, blood plasma, serum, lymph, bone marrow fluid, saliva, eyeball fluid, spermatic fluid, brain extract, spinal fluid, joint fluid, thymic fluid, ascitic fluid and amnion, but not limited to. Preferably, the sample useful in the present invention is virus, bacterium, cell or tissue-derived extract, lysate or purified materials, blood, blood plasma, serum or lymph. Most preferably, the sample is virus, bacterium cell or tissue-derived extract or lysate (for example red blood cell lysaste).

The most important component in the present measurement system is a capturing agent and a detection antibody.

The term “capturing agent” used herein means any material which can capture and immobilize the enzyme to be analyzed. The capturing agent can be any material which has the binding affinity to the enzyme to be analyzed. In addition, the capturing agent may be or not be specific to the enzyme. Preferably, the capturing agent has the binding affinity and specificity to the enzyme. Non-limiting examples of the capturing agent include antibody, receptor, streptavidin (or avidin), aptamer, lectin, DNA, RNA, ligand, coenzyme, inorganic ion, cofactor, sugar, lipid and substrate. Preferably, the capturing agent is antibody, streptavidin (or avidin) or aptamer. More preferably, the capturing agent is an antibody. Still more preferably, the capturing agent is an antibody having the binding capacity and specificity to the enzyme. Most preferably, the capturing agent is a monoclonal antibody having the binding capacity and specificity to the enzyme.

In the present invention, the capturing agent is immobilized on the surface of the solid matrix. The suitable matrix on the surface of which the capturing agent is immobilized is any material which can be used in the art the present invention pertains to. For example, the matrix includes hydrocarbon polymers such as polystyrene and polypropylene, glass, metals and gels, but not limited to. The solid matrix may be provided as the form of dipstick, microtiter plate, particle (e.g., bead), an affinity column and an immunoblot membrane (e.g., polyvinylidene fluoride membrane), protein chips or test tube, cuvette (see U.S. Pat. Nos. 5,143,825, 5,374,530, 4,908,305 and 5,498,551). Most preferably, the solid matrix is a microtiter plate.

The material to be analyzed in the present invention is an enzyme. The suitable enzyme, but not limited to, includes an oxidizing-reducing enzyme, transferases, hydrolases, lyases, isomerases and ligases.

According to the present invention, the enzyme is bound to the capturing agent by contacting to the capturing agent the sample containing the enzyme of interest. As samples, purified materials obtained through purification procedures may be utilized. However, the method of the present invention permits the activity and amount of enzymes to be precisely analyzed even when non-purified bio-samples (e.g., cell-derived extract or lysate) are directly incubated with the capturing agent.

Where an enzyme to be analyzed binds to the capturing agent, the enzyme exhibits its activity in the step (b). The measurement of the activity of the enzyme bound to the capturing agent can be carried out by suitable protocols for the enzyme in accordance with well known methods for measuring the activity of an enzyme (see Robert K. Scopes, Protein Purification, 2^{nd} ed. Springer-Verlag New York Inc. (1988)). Generally, the measurement of the activity of an enzyme is carried out by measuring the product which is produced by incubating the substrate with the enzyme captured by the capturing agent. The method for measuring the activity of an enzyme is largely divided into two methods: stopped methods and continuous methods. The continuous methods include uncoupling methods and coupling methods.

For example, the measurement of the enzyme activity includes (i) a direct or indirect analysis method using various indicators, and (ii) a coupling analysis method using indicator enzymes and indicators.

In the method (i), the indicator compound reacts directly or indirectly with an enzyme reaction product. The example for the method (i) includes a method for detecting fatty acid using rhodamine B in lipase analysis (see U.S. Pat. No. 5,914,245) and a method for detecting proton, electron, ion transfer and pH change that are produced during enzyme reactions by using dyes (e.g., fluoroscein, Oregon Green, Rhodole, thymol blue, methyl orange, and bromocrezol blue).

The measurement of the activity of an enzyme which does not generate chromogenic or fluorescent product can be carried out by coupling an additional enzyme and indicator that can produce detectable product. In addition, the coupling analysis method may be used in order to amplify the activity of an enzyme to be analyzed. For example, even though an enzyme to produce chromogenic products such as NADH and NADPH is analyzed, the activity of the enzyme can be analyzed by using a coupling enzyme that can produce a stronger chromogenic product with precipitation (e.g., Formarzan).

According to a specific example of the present invention, the measurement of glutathione peroxidase activity is carried out by coupling analysis method. Oxidative glutathione (GSSG) produced by glutathione peroxidase is reduced by a coupling enzyme, glutathione reductase and NADP⁺ is then detected at 340 nm on spectrophotometers, thereby indirectly measuring the activity of glutathione peroxidase.

After measuring the activity of the enzyme captured by the capturing agent, the amount of the enzyme is then measured in the same measurement system, that is, in in situ manner. After materials used for analyzing the activity of the enzyme are removed (preferably by washing), the enzyme captured by the capturing agent is contacted to a detection antibody specific to the enzyme to form an antigen-antibody complex. Afterwards, the amount of the antigen-antibody complex formed is measured.

The detection antibody used in the present invention has the binding specificity to the enzyme to be analyzed, and may be a polyclonal antibody or a monoclonal antibody, preferably, monoclonal antibody. Where an antibody is used as the capturing agent, the detection antibody should bind to different epitopes from the one to which the antibody of the capturing agent binds.

The antibody specific to an enzyme can be produced by technologies conventionally performed in the art, for example, fusion method (Kohler and Milstein, European Journal of Immunology, 6:511-519(1976)), recombinant DNA technology(U.S. Pat. No. 4,816,567) or phage antibody library method (Clackson et al, Nature, 352:624-628(1991); and Marks et al, J. Mol. Biol., 222:58, 1-597(1991)).

General procedures for producing an antibody can be found in Harlow, E. and Lane, D., Using Antibodies: A Laboratory Manual, Cold Spring Harbor Press, New York, 1999; Zola, H., Monoclonal Antibodies, A Manual of Techniques, CRC Press, Inc., Boca Raton, Fla., 1984; and Coligan, CURRENT PROTOCOLS IN IMMUNOLOGY, Wiley/Greene, N.Y., 1991, which are incorporated herein by references.

For example, the preparation of hybridoma cell lines for monoclonal antibody production is done by fusion of an immortal cell line and the antibody producing lymphocytes. This can be done by techniques well known in the art.

Polyclonal antibodies of this invention may be prepared by injection of the enzyme antigen to suitable animal, collecting antiserum containing antibodies from the animal, and isolating specific antibodies by any of the known affinity techniques. Even if the detection or measurement of antigen-antibody complex formation can be carried out by directly detecting the antigen-antibody complex, it is preferable that the detection or measurement is carried out by using a label which can produce a detectable signal. According to a preferred embodiment of the present invention, the detection antibody is bound to a label which can generate a detectable signal, or bound to affinity materials. The label includes, but not limited to, an enzymatic (e.g., alkaline phosphatase, β-galactosidase, horseradish peroxidase, β-glucosidase and Cytochrome P₄₅₀), a radioactive (e.g., C¹⁴, I¹²⁵, p³² and S³⁵), a fluorescent (e.g., fluorescein), a luminescent, a chemiluminescent and a FRET (fluorescence resonance energy transfer) label. For the easy detection, the affinity materials, which can be bound to the detection antibody, include biotin. Various labels and methods for labeling antibodies are described in Ed Harlow and David Lane, Using Antibodies, A Laboratory Manual, Cold Spring Harbor Laboratory Press, 1999, which is incorporated herein by references.

If a radioactive isotope is used as the label bound to the detection antibody, the antigen-antibody complex formed in the last step of this invention can be detected by measuring the radioactivity.

If an enzyme catalyzing a chromogenic reaction is used as a label bound to the detection antibody, the antigen-antibody complex can be detected by adding the substrate usable in enzyme-substrate reaction into the measuring system of the present invention and by measuring the reaction product. For example, if an alkaline phosphatase is used as a label, chromogenic reaction substrates such as bromochloroindolylphosphate (BCIP), nitro blue tetrazolium (NBT), naphthol-AS-B1-phosphate and enhanced chemifluorescence (ECF) may be used; and if an horseradish peroxidase is used as a label, substrates such as chloronaphtol, aminoethylcarbazol, diaminobenzidine, D-luciferin, lucigenin (bis-N-methylacridinium nitrate), resorufin benzyl ether, luminol, Amplex Red reagent (10-acetyl-3,7-dihydroxyphenoxazine), TMB (3,3,5,5-tetramethylbenzidine), ABTS (2,2-Azine-di[3-ethylbenzthiazoline sulfonate]) and o-phenylenediamine (OPD) may be used.

Where the detection antibody is bound to affinity materials, the antigen-antibody complex can be detected by using binding partners conjugated with a label (e.g., chromogenic reaction catalyzing enzymes) to generate a detectable signal. For example, where a biotin-conjugated detection antibody is used, the antigen-antibody complex can be detected by measuring the signal generated from chromogenic substrates for streptavidin-conjugated color-developing reaction catalyzing enzymes (e.g., horseradish peroxidase) incubated with the antigen-antibody complex.

According to a preferred embodiment of the present invention, the instant method is used to analyze the enzyme that catalyzes an oxidative-reductive reaction. The specific example of the oxidative-reductive enzyme to be analyzed includes glutathione peroxidase and thioredoxin.

According to a preferred embodiment of this invention, the present method is performed to analyze the activity and amount of glutathione peroxidase, more preferably glutathione peroxidase 1 (GPx1), most preferably human glutathione peroxidase 1.

Oxidative stress has been implicated in aging and phathogenesis of a number of disorders. Oxidative stress may be defined as an imbalance between the production and degradation of reactive oxygen such as superoxide anion, hydrogen peroxide, lipid peroxides, and peroxynitrite. Enzymatic inactivation of reactive oxygen species is achieved mainly by glutathione peroxidase, superoxide dismutase, and catalase(1). The extent of injury by reactive oxygen is related to increase or decrease of one or more of free radical scavenging enzymes of which glutathione peroxidase (GPx) is one. In mammalian cells, glutathione peroxidases constitute the principal antioxidant defense system (2, 3). Various diseases show different levels of the universally present GPx in all tissues. In mice, glutathione peroxidase 1 deficiency results in abnormal vascular and cardiac function and structure (4). GPx 1 activities were described in liver, lungs, platelets, and erythrocyte (5, 6).

According to a specific example of the present invention to measure the activity and amount of glutathione peroxidase (preferably, GPx1), the present invention can be carried out as follows:

First, a sample (preferably, erythrocytes lysate) is contacted to a glutathione peroxidase-specific antibody (capturing agent) which is coated on the surface of the microtiter plate. Then, the activity of the glutathione peroxidase bound to the capturing agent is measured.

It is preferable that the measurement of the activity of glutathione peroxidase is carried out in accordance with a coupling analysis method. Oxidative glutathione (GSSG) which is produced by the catalytic activity of glutathione peroxidase, is reduced by a coupling enzyme, glutathione reductase, and NADP⁺ is produced during this process. The glutathione peroxidase activity is indirectly measured by determining the absorbance of NADP⁺ at 340 nm with spectrophotometer. The rate of decrease in the absorbance at 340 nm is directly proportional to the activity of glutathione peroxidase in the sample.

Afterwards, the microtiter plate is washed and incubated with detection antibody, after which the amount of the glutathione is measured by detecting the antigen-antibody complex formed.

According to a preferred embodiment, the present method is carried out to measure the activity and amount of thioredoxin, preferably thioredoxin 1 (Trx 1), most preferably human thioredoxin 1. According to a specific example of the present invention, the present invention can be carried out as below:

First, a sample is contacted to a thioredoxin-specific antibody (a capturing agent) coated on the surface of the microtiter plate. Then, the activity of the thioredoxin bound to the capturing agent is measured.

Preferably, the measurement of the activity of thioredoxin is carried out in accordance with a coupling analysis method. For example, a sample of interest is allowed for reacting with a protein reducible by thioredoxin in the presence of a reducing agent (DTT, dithiothreitol), resulting in a formation of a reduced protein. Then, the thioredoxin activity can be analyzed by measuring A_{650 nm} of the reduced protein. The microtiter plate is washed and in turn the thioredoxin amount is measured by detecting the antigen-antibody complex formed.

In another aspect of this invention, there is provided an in situ measurement kit for measuring the activity and amount of an enzyme in a sample in a single measurement system, which comprises; (i) a solid matrix, (ii) a capturing agent having the capacity to bind to the enzyme to be analyzed and immobilized on the solid matrix, (iii) a substrate for the enzyme to be analyzed, and (iv) a detection antibody specifically binding to the enzyme to be analyzed.

The kit of this invention is one for embodying the method of the present invention, and common features between these two inventions will be omitted to avoid undue complexity of the specification.

Where the kit of the present invention is fabricated to measure enzyme activity by a coupling analysis method, it may further comprise coupling enzymes and substrates for coupling reactions. Where a chromogenic reaction-catalyzing enzyme binds to detection antibodies, the kit of the present invention may further comprise a substrate for this enzyme. Where biotin binds to the detection antibody, the kit of the present invention may further comprise streptavidin (or avidin) conjugated with a chromogenic reaction catalyzing enzyme. The kit of the present invention can be manufactured as a number of separate packagings and compartments.

According to conventional technologies, separate measurement systems are generally used to measure the activity and amount of an enzyme. However, the present invention permits to measure the activity and amount of an enzyme in a single measurement system in in situ manner (or simultaneous manner). That is, the present invention could get rid of needs for separate measurement system to measure both activity and amount of an enzyme. Where the activity and amount of an enzyme is measured by separate systems, the values measured cannot be considered for the same or identical samples and enzymes because the activity and amount of an enzyme are measured in a spatially and timely separate manner. However, the present invention can give the accurate activity and amount data of an enzyme simultaneously because the measurement of enzyme activity and amount is carried out in a single and same measurement system for the same sample. In addition, the present invention enables to analyze enzymes with fewer amounts in a convenient and accurate manner.

### BRIEF DESCRIPTION OF THE DRAWINGS

FIG. 1a is a concentration standard curve of glutathione peroxidase 1 (GPx1) measured by using the kit of the present invention.

FIG. 1b shows the activities of glutathione peroxidase 1 (GPx1) in erythrocytes measured in accordance with the method of the present invention. RBCs hemolysates with various dilution folds were used as a sample.

FIG. 1c shows the amounts of GPx1 in erythrocytes measured in accordance with the method of the present invention. RBCs hemolysates with various dilution folds were used as a sample.

FIG. 2a is a concentration standard curve of thioredoxin 1 (Trx 1) measured by using the kit of the present invention.

FIG. 2b shows the activities of thioredoxin 1 (Trx 1) in erythrocytes measured in accordance with the method of the present invention.

FIG. 2c shows the amounts of Trx 1 in erythrocytes measured in accordance with the method of the present invention.

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Example 1

### Materials and Methods

### Materials

A UV-spectrophotometer (Molecular device) and a water deionization system (Mirae ST) were used. β-NADPH (Sigma N7505); glutathione peroxidase (LabFrontier, Cat. No. LF-P0071); reduced glutathione (Sigma G4251); t-butylated hydroperoxide (t-BHP) (Sigma B2633); Na₂EDTA (USB) were used. Dilution solvents: 0.1M Tris buffer/1 mM EDTA (pH 7.0). Initiator: 1 mM t-BHP.

### Preparation of Erythrocytes

Blood was collected using EDTA as the anticoagulant. The RBC was spin down by centrifugation at 3000 rpm for 10 min at 4° C. The buffy coat (the white interference between the pelleted RBCs and the plasma) was removed and discarded. The RBC pellet was washed with 10 volumes of cold saline at 4° C., and centrifuged at 3000 rpm for 10 min at 4° C. The clear saline was discarded from top. This was repeated once more. The RBCs was lysed by adding 10 volumes of cold deionized water to the packed cells. If they will be assayed the same day, they are storee on ice, otherwise, frozen at −70° C. The concentration of lysate was determined to determine the appropriate dilution fold.

### Preparation of LF-GPx1 Activity ELISA Kit

LF-GPx1 activity ELISA Kit of this invention which can measure glutathione peroxidase 1 (Gpx 1) activity and amount in in situ manner was prepared based on a sandwich Enzyme-Linked Immunosorbent Assay(ELISA). The 96 well plate has been pre-coated with a monoclonal antibody (LabFrontier, Cat. No. LF-MA0090) specific to human GPx1 (capture antibody). Biotin-conjugated monoclonal antibody specific to human GPx1 (LabFrontier, Cat. No. LF-MA0091) as a detection antibody, HRP (horseradish peroxidase)-conjugated Immunopure Avidin (Pierce) and TMB (3,3,5, 5-tetramethylbenzidine; Bio-Fx, TMBC-1000-01) have been included in the Kit. In addition, to measure Gpx 1 activity, NADPH (Sigma, N7505), GSH (Sigma, G4251), GSSG reductase (LabFrontier, LF-P00071, Yeast form) and t-butylated hydroperoxide have been included in LF-GPx1 activity ELISA Kit.

### Erythrocyte GPx1 Activity Assay in LF-GPx1 Activity ELISA

The glutathione peroxidase-like activity of the compounds under study was assessed by their ability to catalyze a reaction between hydroperoxides and glutathione in an aqueous buffer at physiological pH. The oxidation of GSH to GSSG was measured indirectly by spectrophotometrically assessing the stimulated oxidation of NADPH in the presence of glutathione reductase. Incubations were conducted at room temperature in a Molecular device spectrophotometer recording at 340 nm. Reaction in LF-GPx1 activity ELISA Kit was made with 100 mM Tris buffer (pH 7.0). First, RBC hemolysate (5.5 μl) obtained in the preceding procedure was added into the Kit, and NADPH (0.2 mM), GSH (1 mM), GSSG reductase (0.2 unit), hydroperoxide (1 mM) were further added. And then, the decline in absorbance was recorded at 340 nm. Rate assessments were performed when the decline in absorbance was constant for at least 20 minutes. 1 unit of GPx1 is defined as the amount of an enzyme that can oxidize 1 nmole of NADPH to NADP⁺ at 25° C.

### Quantification of Erythrocyte GPx1 Using LF-GPx1 Activity ELISA

The plate was washed with PBST to remove all substrates after enzyme activity assay. 100 μl of biotinylated-monoclonal antibody (2 μg/ml) specific to human GPx1 was added and incubated at 37° C. for 60 minutes. And then, the plate was washed with PBST, treated with 100 ml of HRP-conjugated Immunopure Avidin (0.5 μg/ml), treated with 100 ml of TMB, and allowed it to produce signals by incubating it at 25° C. for 3 minutes. The enzyme-substrate reaction was terminated by the addition of 100 μl of 1N H₂SO₄ and measured the absorbance at 450 nm. To calculate the GPx1 concentrations from the absorbance observed, GPx1 concentration standard curve (0-100 ng/ml) was obtained in the assay.

### Results

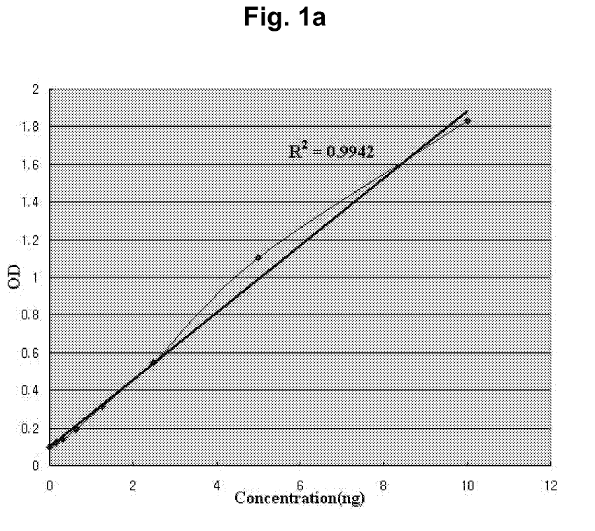

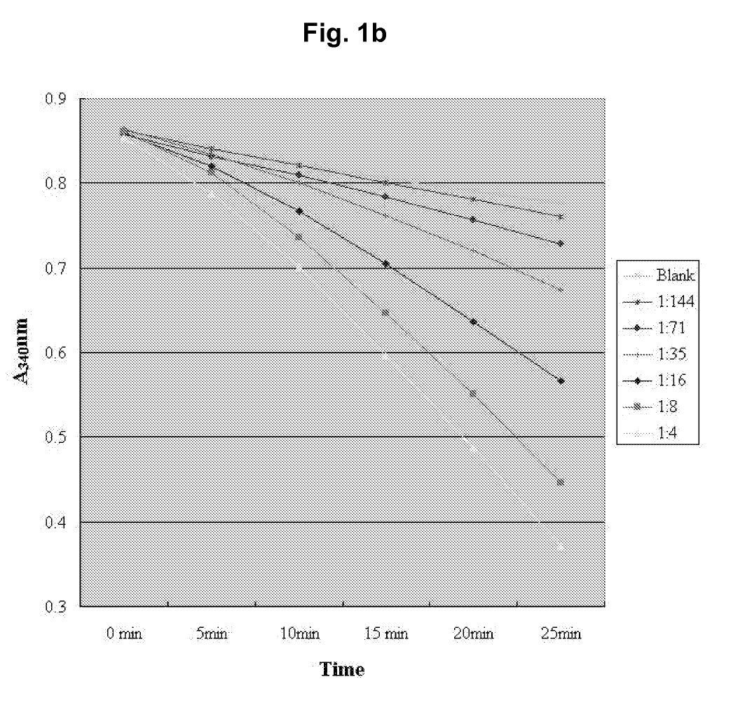

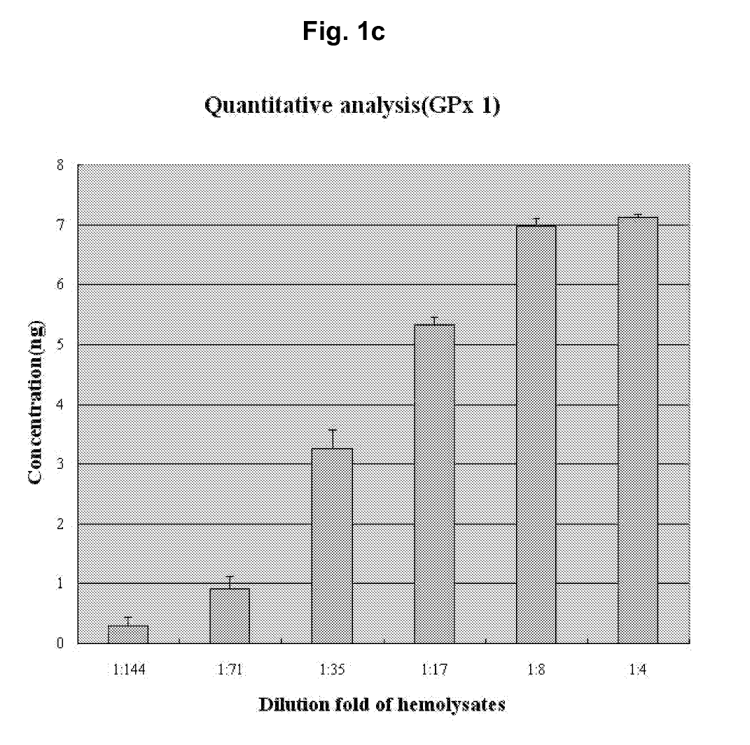

FIGS. 1b and 1c show GPx1 activities and quantities in human RBC hemolysates, which were measured using the LF-GPx1 activity ELISA kit of this invention. Experiments have been performed with various dilutions of RBC hemolysate. Activity is expressed as mU/mg hemolysate. As shown in FIG. 1b, the kit of this invention gave accurate analysis data in the senses that it shows increasing values in a concentration-dependent manner over a range from 1:4 to 1:144 (the range of serial 36-fold dilution) of the hemolysate. Analysis of the results of experiment reveals that GPx 1 activities in dilutions of 1:4, 1:8, 1:16, 1:35, 1:71 and 1:144 corresponds to 2.94 mU/mg, 2.49 mU/0.5 mg, 1.79 mU/0.25 mg, 1.15 mU/0.125 mg, 0.81 mU/0.0625 mg and 0.65 mU/0.03125 mg, respectively. In FIG. 1c, the dilutions of 1:4, 1:8, 1:16, 1:35, 1:71 and 1:144 of the RBC hemolysate corresponds 1 mg/0.1 ml, 0.5 mg/0.1 ml, 0.25 mg/0.1 ml, 0.125 mg/0.1 ml, 0.062 mg/0.1 ml and 0.031 mg/0.1 ml RBC hemolysate, respectively.

### Example 2

### Materials and Methods

### Materials

A UV-spectrophotometer (Molecular device) and a water deionization system (Mirae ST) were used. Insulin (Sigma I5500); human thioredoxin (LabFrontier, Cat. No. LF-P0001); DTT (USB) were used. Dilution solvents: 50 mM Tris buffer/1 mM EDTA (pH 7.5). Initiator: 1.6 mM DTT(dithiothreitol).

### Preparation of LF-Trx 1 Activity ELISA Kit

LF-Trx 1 activity ELISA Kit of the present invention which can measure thioredoxin 1 (Trx 1) activity and amount in in situ manner was prepared based on a sandwich Enzyme-Linked Immunosorbent Assay(ELISA). The 96 well plate has been pre-coated with a monoclonal antibody (LabFrontier, Cat. No. LF-MA0050) specific to human Trx 1 (capture antibody). Biotin-conjugated monoclonal antibody specific to human Trx 1 (LabFrontier, Cat. No. LF-MA0077) as a detection antibody, HRP-conjugated Immunopure Avidin (Pierce) and TMB have been included in the Kit. In addition, to measure Trx 1 activity, DTT, Insulin (Sigma, I5500) and recombinant human thioredoxin (LabFrontier, Cat. No. LF-P0001) have been included in LF-Trx 1 activity ELISA Kit.

### Recombinant Human-Trx 1 Activity Assay in LF-Trx 1 Activity ELISA

The thioredoxin-like activity under this study was assessed by measuring O.D. of Insulin-(SH)₂, which have been generated by the catalysis of the thioredoxin under the presence of reductive DTT, through spectrophotometer at 650 nm. The increasing rate of O.D. at 650 nm is directly proportional to the activity of thioredoxin in the sample. Incubations were conducted at room temperature in a Molecular device spectrophotometer recording at 650 nm.

Reaction in LF-Trx1 activity ELISA Kit was made with 50 mM Tris buffer (1 mM EDTA, pH 7.5). First, 100 μl of recombinant thioredoxin protein (serial dilutions from 5 ng), 1.6 mM DTT and 200 μg of insulin were added to the Kit, and incubated at 25° C. And then, the increase in O.D. at 650 nm was recorded. Rate assessments were performed when the increase in O.D. was constant for at least 30 minutes. 1 unit of Trx 1 is defined as the amount of an enzyme that can change 1 A₆₅₀ per 1 minute at 25° C. in the above insulin reduction assay.

### Quantification of Recombinant Human-Trx 1 Using LF-Trx 1 Activity ELISA

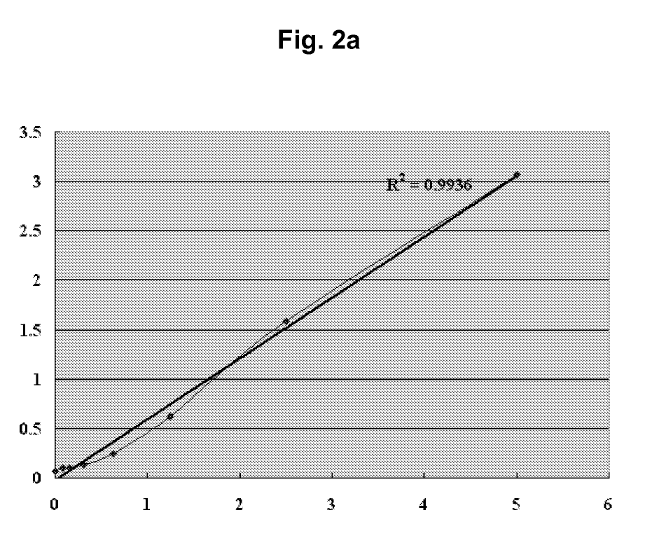

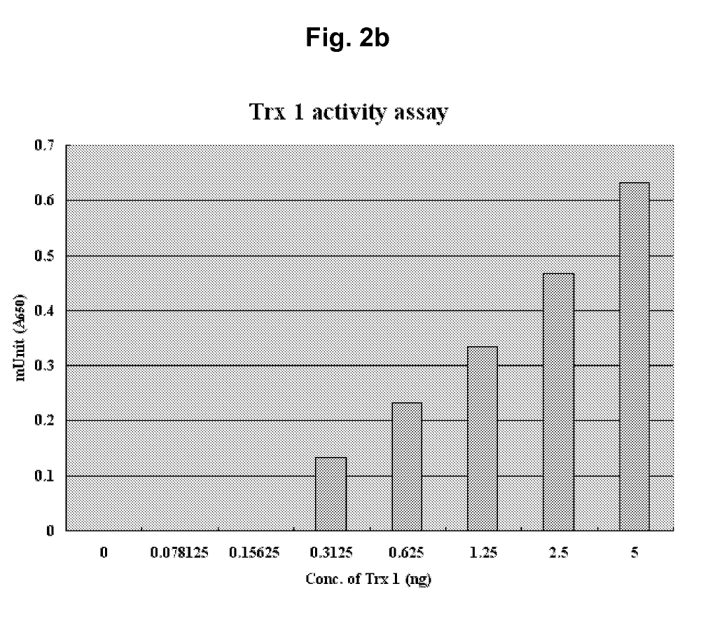

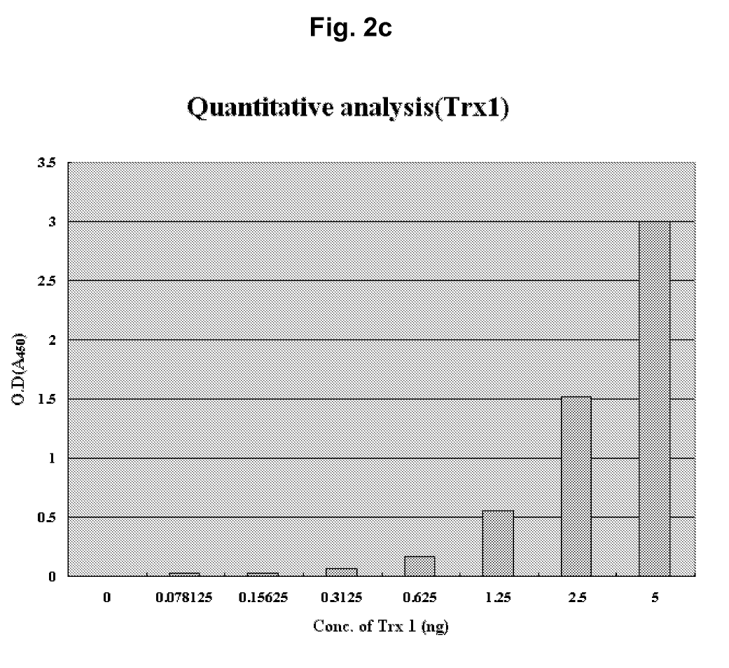

The plate was washed with PBST to remove all substrates and DTT after enzyme activity assay. 100 μl of biotinylated-monoclonal antibody (0.5 μg/ml) specific to human TRx 1 was added and incubated at 37° C. for 60 minutes. And then, the plate was washed with PBST, treated with 100 ml of HRP-conjugated Immunopure Avidin (0.5 μg/ml), treated with 100 ml of TMB, and allowed it to produce signals by incubation at 25° C. for 3 minutes. The enzyme-substrate reaction was terminated by the addition of 100 μl of 1N H₂SO₄ and measured the absorbance at 450 nm. To calculate the Trx 1 concentrations from the absorbance observed, TRx 1 concentration standard curve (0-50 ng/ml) were obtained in the assay (FIG. 2a).

### Results

FIGS. 2b and 2c show Trx1 activities and quantities measured using the LF-Trx 1 activity ELISA kit of this invention. Experiment has been performed with various dilutions of recombinant Trx 1. Activity is expressed as U/mg. As shown in FIG. 2b, the kit of this invention gave accurate analysis data in the senses that it shows increasing values in a concentration-dependent manner over a range from 5 ng to 0.078 ng of recombinant Trx 1. Analysis of the result of experiments reveals that Trx 1 activities in concentrations of 5, 2.5, 1.25, 0.625 and 0.3125 ng correspond to 38, 56, 80, 112 and 128 U/mg, respectively. As shown in FIG. 2c, the O.D. values increase as the concentration of Trx 1 increases.

Therefore, it could be understood that the kit of the present invention permits to precisely measure both activity and amount of the enzyme in a single measurement system.

The present invention provides methods and kits for measuring in situ the activity and amount of the enzyme in a single measurement system. According to the present invention, there is no need for separate systems. In addition, the present invention permits to measure the precise enzyme activity and amount because the measurement is carried out in a single measurement system as to the same sample with in almost simultaneous manner. In addition, the present invention permits to analyze the existence of an enzyme with higher specificity and sensitivity in more convenient manner.

Having described a preferred embodiment of the present invention, it is to be understood that variants and modifications thereof falling within the spirit of the invention may become apparent to those skilled in the art, and the scope of this invention is to be determined by appended claims and their equivalents.

### REFERENCES

1. Forsberg L, de Faire U, Morgenstern R. Oxidative stress, human genetic variation, and disease. Arch Biochem Biophys. 2001 May 1;389(1):84-93.
2. Ursini F, Maiorino M, Brigelius-Flohe R, Aumann K D, Roveri A, Schomburg D, Flohe L. Diversity of glutathione peroxidases. Methods Enzymol. 1995;252:38-53. No abstract available.
3. Raes M, Michiels C, Remacle J. Comparative study of the enzymatic defense systems against oxygen-derived free radicals: the key role of glutathione peroxidase. Free Radic Biol Med. 1987;3(1):3-7.
4. Forgione M A, Cap A, Liao R, Moldovan N I, Eberhardt R T, Lim C C, Jones J, Goldschmidt-Clermont P J, Loscalzo J. Heterozygous cellularglutathione peroxidase deficiency in the mouse: abnormalities in vascular and cardiac function and structure. Circulation. 2002 Aug. 27;106(9):1154-8.
5. Atroshi F, Sankari S, Osterberg S, Sandholm M. Variation of erythrocyte glutathione peroxidase activity in Finn sheep. Res Vet Sci. 1981 Nov.;31(3):267-71.
6. Doni M G, Avventi G L, Bonadiman L, Bonaccorso G. Glutathione peroxidase, selenium, and prostaglandin synthesis in platelets. Am J Physiol. 1981 May;240(5):H800-3.
7. Holmgren A. Thioredoxin catalyzes the reduction of insulin disulfides by dithiothreitol and dihydrolipoamide. J Biol Chem. 1979 Oct. 10;254(19):9627-32.

What is claimed is:

1. A method for measuring in situ the activity and amount of an enzyme in a single measurement system, which comprises the steps of:
(a) contacting a sample to a capturing agent having the capacity to bind to the enzyme to be analyzed and immobilized on a solid matrix;
(b) measuring the activity of the enzyme captured by the capturing agent;
(c) contacting a detection antibody specific to the enzyme captured by the capturing agent; and
(d) detecting an antigen-antibody complex formed in the step (c).

2. The method according to claim 1, wherein the sample is selected from the group consisting of virus, bacterium, cell or tissue-derived extract, lysate or purified material, blood, blood plasma, serum, lymph, bone marrow fluid, saliva, eyeball fluid, spermatic fluid, brain extract, spinal fluid, joint fluid, thymic fluid, ascitic fluid and amnion.

3. The method according to claim 1, wherein the capturing agent is selected from the group consisting of antibody, receptor, streptavidin, avidin, aptamer, lectin, DNA, RNA, ligand, coenzyme, inorganic ion, cofactor, sugar, lipid and substrate.

4. The method according to claim 1, wherein the capturing agent is antibodies.

5. The method according to claim 1, wherein the detection antibody is bound to a label to generate a detectable signal or bound to affinity materials.

6. An in situ measurement kit for measuring the activity and amount of an enzyme in a single measurement system, which comprises (i) a solid matrix, (ii) a capturing agent having the capacity to bind to the enzyme to be analyzed and immobilized on the solid matrix, (iii) a substrate for the enzyme to be analyzed, and (iv) a detection antibody specifically binding to the enzyme to be analyzed.
